(19) United States
(12) Patent Application Publication
(10) Pub. No.: US 2003/0148971 A1
(43) Pub. Date: Aug. 7, 2003

(54) Treatment of inflammatory and malignant diseases

(76) Inventors: Malcolm Lovell Handel, Lindfield (AU); Ly Quoc Quynh Nguyen, Randwick (AU); David G. Atkins, New York, NY (US); Murray John Cairns, Woy Woy (AU)

Correspondence Address: MORRISON & FOERSTER LLP, 755 PAGE MILL RD, PALO ALTO, CA, 94304-1018, US

(21) Appl. No.: 10/068,471

(22) Filed: Feb. 4, 2002

(51) Int. Cl. A61K 48/00; C07H 21/02

(52) U.S. Cl. 514/44; 536/23.1

(57) ABSTRACT

The present invention relates to DNAzymes which are targeted against mRNA molecules encoding RelA(p65) (a subunit of NF-κB). The present invention also relates to compositions including these DNAzymes and to methods of treatment involving administration of the DNAzymes.

### FIELD OF THE INVENTION

The present invention relates to DNAzymes which are targeted against mRNA molecules encoding a subunit of the transcription factor NF-κB. The present invention also relates to compositions including these DNAzymes and to methods of treatment involving administration of the DNAzymes.

### BACKGROUND OF THE INVENTION

### Arthritis

Arthritis research in recent times has largely focused on the discovery of inhibitors of individual mediators of inflammation, particularly, inhibitors of TNFα and IL-1β. A potential inadequacy of this approach is that there is a large number of gene products that act as mediators of inflammation and the inhibition of any one, or even several, of the mediators of inflammation may be insufficient to fully control the course of rheumatoid arthritis (RA). This is illustrated by the failure to control joint erosion by inhibition of cyclo-oxygenases with non-steroidal anti-inflammatory drugs. Inhibition of TNFα or IL-1β promise to have more profound benefits than cyclo-oxygenase inhibition, however, the inhibition of many mediators of inflammation may be required for complete control of RA. Transcription factors, which bind the promoter regions of genes to induce their expression at the level of mRNA synthesis, are capable of simultaneous control of many mediators of inflammation. A transcription factor which is necessary for the expression of a large number of mediators of inflammation is therefore a suitable target in the therapy of RA.

### Transcription Factor NF-κB in Arthritis

The inducible transcription factor NF-κB, typically a heterodimer of p50 and RelA(p65), is particularly important in the regulation of gene expression in inflammation. Inducers of NF-κB include TNFα, IL-1β, PDGF, oxidative stress, viral products and bacterial cell wall products such as LPS. In turn, NF-κB can activate the transcription of cytokines (TNFα, IL-1β, IL-6, IL-8), adhesion molecules (ICAM-1, VCAM-1, E-selectin) and enzymes (iNOS, COX-2, cPLA₂) that form the main known contributors to the inflammatory process. NF-κB transcriptional activity is largely controlled by sequestration of NF-κB in the cytoplasm by a family of proteins, IκBs. Upon stimulation of the cell IκB is degraded leading to translocation of NF-κB to the nucleus where it binds the promoter sequences of numerous genes, such as those listed above. Since NF-κB is localised in the nuclei of synovial cells in RA (Handel et al, 1995a) and the list of inducers and targets of NF-κB very closely match the profile of inflammatory mediators in RA, an important role for activated NF-κB in human RA is likely. This is supported by animal models in which NF-κB decoys and an IκB repressor effectively reduced streptococcal cell wall-induced and pristane-induced arthritis in rats (Miagkov et al, 1998).

Another transcription factor, AP-1, may also be important in the pathogenesis of inflammatory arthritis. AP-1 is localised in the nuclei of fibroblast-like CD14-negative type B synovial lining cells (Handel et al, 1995a). AP-1 is important for the expression of metalloproteinases, especially collagenase and stromelysin, that are likely to contribute to the erosion of bone and cartilage in RA (Brinckerhoff,1991).

It should be noted that NF-κB is found predominantly in macrophages, although it is also present in a subset of fibroblasts. In contrast, AP-1 is found almost exclusively in synovial lining fibroblasts (Handel et al, 1995a; Kinne et al, 1994). It is proposed that a hierarchy exists, whereby NF-κB activity in macrophages (Type A synovial cells) is responsible for AP-1 activation in neighbouring fibroblasts (Type B synovial cells). The basis for this hypothesis begins with the observation that expression of TNFα is predominantly confined to synovial lining macrophages (Chu et al, 1991), presumably through the activity of NF-κB. TNFα has already been placed at the head of a hierarchy of cytokines, particularly because TNFα controls IL-1β and IL-6 expression in synovial cells, and not vice versa. Moreover, metalloproteinase expression by synovial fibroblasts has clearly been shown to be induced by TNFα and IL-1β. As mentioned above, metalloproteinase expression is AP-1 dependent, or in other words, the expression of an AP-1 dependent gene in fibroblasts is due to the effect of a cytokine, namely TNFα, which is NF-κB dependent in macrophages. The hypothesis that NF-κB activity in macrophages is of primary importance is further supported by the observation that joint erosion in RA correlates with the density of macrophages in the synovium.

### NF-κB in Cancer and Apoptosis

NF-κB plays a role in the fundamental processes of cell proliferation and apoptosis. Chemotherapy and radiation in some cancer cells can induce NF-κB activity. Activation of NF-κB protects against apoptosis therefore leading to resistance to these therapies. Inhibition of NF-κB by antisense oligonucleotides or by expression of its inhibitor I-κBa has been observed to cause tumour regression in adult T-cell leukemia (Kitajima, 1992) and human breast carcinomas (Higgins, 1993; Cai, 1997) amongst other tumours. More recently it has been shown that inhibition of NF-κB overcomes resistance to chemotherapy in a model of fibrosarcoma through increased apoptosis (Wang, 1999). It is reasonable to hypothesise that inhibition of NF-κB will result in regression and/or chemosensitivity in a wide variety of cancers and leukaemias.

### Inhibitors of NF-κB

Several existing drugs have actions that directly, or indirectly, inhibit NF-κB and/or AP-1. These include glucocorticosteroids, retinoids, gold thiolates and D-penicillamine. Salicylates as well as chloroquine and the other aminoquinolines may also have indirect effects on NF-κB. Another transcription factor, NF-AT, is indirectly inhibited by cyclosporine and tacrolimus (FK506). This list of drugs includes a significant proportion of the useful anti-rheumatic agents, highlighting the importance of transcription factor inhibition as a means of treating rheumatic diseases. An analysis of their mechanisms of action, with reference to their effects on AP-1 and NF-κB, suggests that a selective inhibitor of NF-κB will be safe and effective in the treatment of rheumatoid arthritis.

Glucocorticosteroids: The reliability and effectiveness with which glucocorticosteroids suppress inflammation has meant that they underpin the therapy of many individuals with RA and are extremely useful in crisis situation. Glucocorticosteroids act by binding the intracellular glucocorticoid receptor (GR), a member of the nuclear receptor class of transcription factors. Ligand activated GR can either form homodimers (GR-GR) to up-regulate the expression of genes possessing the GR response element (GRE) or form heterodimers with other transcription factors. Increased expression of GRE dependent genes may be responsible for the development of the main adverse effects of glucocorticosteroids recognized as Cushing's syndrome, although there are so many genes involved that have not been fully characterized that it is difficult to directly attribute all the unwanted metabolic effects to this mechanism. In addition to these metabolic effects of glucocorticosteroids are the anti-inflammatory effects. The metabolic effects, such as obesity, diabetes, cataracts and osteoporosis are the unwanted but unavoidable adverse effects when glucocorticosteroids are used in the treatment of inflammation.

The anti-inflammatory effects of glucocorticosteroids are mediated in large part by inhibition of NF-κB. This is illustrated by studies on the effects of dexamethasone on synovium from the joints of osteoarthritis patients. Using electrophoretic mobility shift analyses (EMSA), DNA binding by NF-κB was induced by TNFα and inhibited by dexamethasone in human synovial tissue explants, clearly demonstrating that glucocorticosteroids are effective inhibitors of NF-κB (Handel et al, 1998). There are several mechanisms by which glucocorticosteroids inhibit NF-κB activity. Ligand activated GR increases the expression of IκBa, an inhibitor that prevents the activation and nuclear translocation of NF-κB (Scheinman et al, 1995; Auphan et al, 1995), although this mechanism does not seem to account for the glucocorticosteroid-induced repression of NF-κB activity in endothelial cells (Brostjan et al, 1996). Another anti-inflammatory mechanism of glucocorticosteroids involves the formation of heterodimers between GR and RelA (p65) resulting in mutual antagonism between glucocorticosteroids and NF-κB activity (Ray and Prefontaine, 1994; Caldenhoven et al, 1995). Competition for limiting amounts of mutually important transcriptional co-factors, particularly p300 and CBP, is another mechanism of mutual antagonism between GR and the pro-inflammatory transcription factors (Kamei et al, 1996).

Gold and D-penicillamine: Gold thiolates and D-penicillamine are thiol reactive drugs. In vitro they interact with thiol groups of cysteine residues in the DNA binding domains of Jun and Fos, thus inhibiting DNA binding of AP-1 (Handel et al, 1995b; 1996). The chemical reactions of these thiol drugs are facilitated by positively charged lysine and arginine residues that flank the cysteine residues of Jun and Fos, thus accelerating the formation of gold-cysteinyl bonds and D-penicillamine-cysteine disulphides. The reaction with D-penicillamine is free radical-dependent whereas the reaction with gold is not. Both reactions are favoured under oxidative conditions of inflammation. The concentration of gold thiomalate required for the inhibition of AP-1 mediated transcription in cultured cells is in the low micromolar range. This concentration range is pharmacologically relevant and is below the concentration reported for the inhibition of any enzyme (Shaw, 1979). Gold thiolates also have similar inhibitory effects on NF-κB (Yang et al, 1995).

Anti-malarial aminoquinolines: Aminoquinolines, including chloroqine and hydroxychloroquine, are basic and they accumulate to very high concentrations in the acidic environment of lysosomes (Poole and Ohkuma, 1981). Acidic sphingomyelinase, which is found in lysosomes and cannot function in the neutralized environment after aminoquinoline treatment, mediates a necessary step in a signal transduction pathway between the p55-TNFα receptor and activation of NF-κB in the nucleus(Weigmann et al, 1994; Schutze et al, 1995). Inhibition of NF-κB is therefore a likely part of the anti-arthritic action of anti-malarial drugs.

Salicylate, NSAIDs and arachidonate: Salicylates have been reported to inhibit NF-κB activation, in addition to their well known effects on cyclooxygenase (Kopp and Ghosh, 1994). The concentration of salicylate required for this effect is very high and the specificity for suppression of NF-κB has been called into question (Frantz and O'Neill, 1995). Of possible mechanistic relevance is the recent observation that arachidonic acid, the precursor of many pro-inflammatory lipids, is directly anti-inflammatory by stabilizing IκB, the inhibitor of NF-κB (Stuhlmeier et al, 1997). It is possible that inhibitors of cyclooxygenase and lipoxygenase may increase intracellular arachidonic acid, providing secondary benefits in the treatment of inflammation via NF-κB inhibition.

Cyclosporin and tacrolimus action: By complexing with immunophilins cyclosporin (CsA) and tacrolimus (FK506) inhibit the activity of calcineurin, thereby blocking the nuclear translocation of nuclear factor (NF-AT). The transcription factor NF-AT is important for the expression of the IL-2 gene, although the relative lack of IL-2 in rheumatoid synovium suggests that CsA action in RA may employ another mechanism. Recently it has become apparent that calcineurin also enhances the degradation of IκB, leading to increased NF-κB DNA binding and transcriptional activity in lymphocytes (Frantz et al, 1994). CsA and tacrolimus therefore have inhibitory effects on both NF-AT and NF-κB, at least in lymphocytes.

Summary of existing drugs: In summary, there are many drugs that have inhibitory effects on NF-κB as their common denominator in the therapy of rheumatoid arthritis. On the basis that the adverse effects of these drugs are all quite different, it seems likely that the adverse effects are not mediated by their common mode of action, suggesting that selective pharmacological inhibition of NF-κB will be both safe and effective.

### DNAzymes

In human gene therapy, antisense nucleic acid technology has been one of the major tools of choice to inactivate genes whose expression causes disease and is thus undesirable. The anti-sense approach employs a nucleic acid molecule that is complementary to, and thereby hybridizes with, an mRNA molecule encoding an undesirable gene. Such hybridization leads to the inhibition of gene expression.

Anti-sense technology suffers from certain drawbacks. Anti-sense hybridization results in the formation of a DNA/target mRNA heteroduplex. This heteroduplex serves as a substrate for RNAse H-mediated degradation of the target mRNA component. Here, the DNA anti-sense molecule serves in a passive manner, in that it merely facilitates the required cleavage by endogenous RNAse H enzyme. This dependence on RNAse H confers limitations on the design of anti-sense molecules regarding their chemistry and ability to form stable heteroduplexes with their target mRNAs. Anti-sense DNA molecules also suffer from problems associated with non-specific activity and, at higher concentrations, even toxicity.

As an alternative to anti-sense molecules, catalytic nucleic acid molecules have shown promise as therapeutic agents for suppressing gene expression, and are widely discussed in the literature (Haseloff (1988); Breaker (1994); Koizumi (1989); Otsuka; Kashani-Sabet (1992); Raillard (1996); and Carmi (1996)). Thus, unlike a conventional anti-sense molecule, a catalytic nucleic acid molecule functions by actually cleaving its target mRNA molecule instead of merely binding to it. Catalytic nucleic acid molecules can only cleave a target nucleic acid sequence if that target sequence meets certain minimum requirements. The target sequence must be complementary to the hybridizing regions of the catalytic nucleic acid, and the target must contain a specific sequence at the site of cleavage.

Catalytic RNA molecules (“ribozymes”) are well documented (Haseloff (1988); Symonds (1992); and Sun (1997)), and have been shown to be capable of cleaving both RNA (Haseloff (1988)) and DNA (Raillard (1996)) molecules. Indeed, the development of in vitro selection and evolution techniques has made it possible to obtain novel ribozymes against a known substrate, using either random variants of a known ribozyme or random-sequence RNA as a starting point (Pan (1992); Tsang (1994); and Breaker (1994)).

Ribozymes, however, are highly susceptible to enzymatic hydrolysis within the cells where they are intended to perform their function. This in turn limits their pharmaceutical applications.

Recently, a new class of catalytic molecules called “DNAzymes” was created (Breaker and Joyce (1995); Santoro (1997)). DNAzymes are single-stranded, and cleave both RNA (Breaker (1994); Santoro (1997)) and DNA (Carmi (1996)). A general model for the DNAzyme has been proposed, and is known as the “10-23” model. DNAzymes following the “10-23” model, also referred to simply as “10-23 DNAzymes”, have a catalytic domain of 15 deoxyribonucleotides, flanked by two substrate-recognition domains. In vitro analyses show that this type of DNAzyme can effectively cleave its substrate RNA at purine:pyrimidine junctions under physiological conditions (Santoro (1997)).

DNAzymes show promise as therapeutic agents. However, DNAzyme success against a disease caused by the presence of a known mRNA molecule is not predictable. This unpredictability is due, in part, to two factors. First, certain mRNA secondary structures can impede a DNAzyme's ability to bind to and cleave its target mRNA. Second, the uptake of a DNAzyme by cells expressing the target mRNA may not be efficient enough to permit therapeutically meaningful results. For these reasons, merely knowing of a disease and its causative target mRNA sequence does not alone allow one to reasonably predict the therapeutic success of a DNAzyme against that target mRNA, absent an inventive step.

### SUMMARY OF THE INVENTION

Accordingly, in a first aspect the present invention provides a DNAzyme which specifically cleaves RelA(p65) mRNA, the DNAzyme comprising

(i) a catalytic domain which cleaves mRNA at a purine:pyrimidine cleavage site;

(ii) a first binding domain contiguous with the 5′ end of the catalytic domain; and

(iii) a second binding domain contiguous with the 3′ end of the catalytic domain,

wherein the binding domains are sufficiently complementary to the two regions immediately flanking a purine:pyrimidine cleavage site within the region of RelA(p65) mRNA corresponding to nucleotides 1 to 1767 as shown in SEQ ID NO: 1, such that the DNAzyme cleaves the RelA(p65) mRNA.

In a second aspect the present invention provides a pharmaceutical composition comprising a DNAzyme of the first aspect and a pharmaceutically acceptable carrier.

In a third aspect, the present invention provides a method of inhibiting NF-κB activity in a cell which method comprises introducing into the cell a DNAzyme of the first aspect.

In a fourth aspect, the present invention provides a method of inhibiting NF-κB activity in a subject which method comprises administering to the subject a pharmaceutical composition of the second aspect.

In a fifth aspect the present invention provides a method of treating an inflammatory disease in a subject which method comprises administering to the subject a therapeutically effective dose of a pharmaceutical composition of the second aspect.

In a sixth aspect the present invention provides a method of treating atherosclerosis in a subject which method comprises administering to the subject a therapeutically effective dose of a pharmaceutical composition of the second aspect.

In a seventh aspect the present invention provides a method of treating cancer or leukaemia in a subject which comprises administering to the subject a therapeutically effective dose of a pharmaceutical composition of the second aspect.

### BRIEF DESCRIPTION OF THE FIGURES

FIG. 1. Effects of DNAzymes ND2 on NF-κB and AP-1 dependent luciferase reporter gene in the presence of a liposome, CellFectin (Life Technologies).

FIG. 2. Effect of DNAzyme (Dz) ND2 compared to its control ND2c on NF-κB dependent transcription.

### DETAILED DESCRIPTION OF THE INVENTION

The present invention provides DNAzymes which specifically target RelA(p65) mRNA and inhibit NF-κB activity.

More specifically, in a first aspect the present invention provides a DNAzyme which specifically cleaves RelA(p65) mRNA, the DNAzyme comprising

(i) a catalytic domain which cleaves mRNA at a purine:pyrimidine cleavage site;

(ii) a first binding domain contiguous with the 5′ end of the catalytic domain; and

(iii) a second binding domain contiguous with the 3′ end of the catalytic domain,

wherein the binding domains are sufficiently complementary to the two regions immediately flanking a purine:pyrimidine cleavage site within the region of RelA(p65) mRNA corresponding to nucleotides 1 to 1767 as shown in SEQ ID NO: 1, such that the DNAzyme cleaves the RelA(p65) mRNA.

In a preferred embodiment of the first aspect of the present invention, the binding domains are entirely complementary to the regions immediately flanking the cleavage site. It will be appreciated by those skilled in the art, however, that strict complementarity may not be required for the DNAzyme to bind to and cleave the RelA(p65) mRNA.

As used herein, “DNAzyme” means a DNA molecule that specifically recognises and cleaves a distinct target nucleic acid sequence, which may be either DNA or RNA.

The catalytic domain of a DNAzyme of the present invention may be any suitable catalytic domain. Examples of suitable catalytic domains are described in Santoro and Joyce (1997) and U.S. Pat. No. 5,807,718. In a preferred embodiment, the catalytic domain has the nucleotide sequence GGCTAGCTACAACGA (SEQ ID NO: 2).

Within the parameters of the present invention, the binding domain lengths (also referred to herein as “arm lengths”) can be of any permutation, and can be the same or different. In a preferred embodiment, the binding domain lengths are at least 6 nucleotides in length, and preferably both binding domains have a combined total length of at least 14 nucleotides. Various permutations such as 7+7, 8+8 and 9+9 are envisioned. It is well established that the greater the binding domain length, the more tightly it will bind to its complementary mRNA sequence. Accordingly, in a further preferred embodiment, each domain is nine or more nucleotides in length.

In a preferred embodiment, the cleavage site corresponds to a site selected from the group consisting of:

(i) the AT site at nucleotides 80-81;

(ii) the GT site at nucleotides 91-92;

(iii) the GT site at nucleotides 140-141;

(iv) the AT site at nucleotides 149-150;

(v) the AT site at nucleotides 215-216;

(vi) the AT site at nucleotides 237-238;

(vii) the AT site at nucleotides 260-261;

(viii)the AT site at nucleotides 350-351;

(ix) the GT site at nucleotides 438-439;

(x) the AT site at nucleotides 479-480;

(xi) the CT site at nucleotides 525-526;

(xii) the GT site at nucleotides 572-572;

(xiii)the AT site at nucleotides 583-584;

(xiv) the AT site at nucleotides 726-727;

(xv) the GT site at nucleotides 734-735;

(xvi) the AT site at nucleotides 749-750;

(xvii) the AT site at nucleotides 807-808;

(xviii) the GT site at nucleotides 830-831;

(xix) the AT site at nucleotides 951-952;

(xx) the GT site at nucleotides 963-964;

(xxi) the AT site at nucleotides 1070-1071;

(xxii) the GT site at nucleotides 1076-1077;

(xxiii) the GT site at nucleotides 1100-1101;

(xxiv) the AT site at nucleotides 1125-1126;

(xxv) the AT site at nucleotides 1175-1176;

(xxvi) the GT site at nucleotides 1235-1236;

(xxvii) the AT site at nucleotides 1279-1280;

(xxviii)the GT site at nucleotides 1307-1308;

(xxix) the GT site at nucleotides 1313-1314;

(xxx) the GT site at nucleotides 1387-1388;

(xxxi) the AT site at nucleotides 1416-1417;

(xxxii) the GT site at nucleotides 1484-1485;

(xxxiii)the GT site at nucleotides 1529-1530;

(xxxiv) the AT site at nucleotides 1553-1554; and

(xxxv) the AT site at nucleotides 1697-1698.

In a particularly preferred embodiment, the cleavage site corresponds to the GT site at nucleotides 91-92.

In a further embodiment, the DNAzyme has a sequence selected from the group consisting of:

(SEQ ID NO:3)

5′ GTTCGTCCAGGCTAGCTACAACGAGGCCGGGGT 3′;

(SEQ ID NO:4)

5′ GAGGGGGAAGGCTAGCTACAACGAAGTTCGTCC 3′;

(SEQ ID NO:5)

5′ TGATCTCCAGGCTAGCTACAACGAATAGGGGCC 3′;

(SEQ ID NO:6)

5′ GCTGCTCAAGGCTAGCTACAACGAGATCTCCAC 3′;

(SEQ ID NO:7)

5′ CGCCTGGGAGGCTAGCTACAACGAGCTGCCCGC 3′;

(SEQ ID NO:8)

5′ TTGGTGGTAGGCTAGCTACAACGACTGTGCTCC 3′;

(SEQ ID NO:9)

5′ TGATCTTGAGGCTAGCTACAACGAGGTGGGGTG 3′;

(SEQ ID NO:10)

5′ CCTTTCCTAGGCTAGCTACAACGAAAGCTCGTG 3′;

(SEQ ID NO:11)

5′ TTCTTCACAGGCTAGCTACAACGAACTGGATTC 3′;

(SEQ ID NO:12)

5′ TGGTCTGGAGGCTAGCTACAACGAGCGCTGACT 3′;

(SEQ ID NO:13)

5′ TAGTCCCCAGGCTAGCTACAACGAGCTGCTCTT 3′;

(SEQ ID NO:14)

5′ GGTCCCGCAGGCTAGCTACAACGATGTCACCTG 3′;

(SEQ ID NO:15)

5′ CCTGCCTGAGGCTAGCTACAACGAGGGTCCCGC 3′;

(SEQ ID NO:16)

5′ ACCTTGTCAGGCTAGCTACAACGAACAGTAGGA 3′;

(SEQ ID NO:17)

5′ CTTTCTGCAGGCTAGCTACAACGACTTGTCACA 3′;

(SEQ ID NO:18)

5′ ACACCTCAAGGCTAGCTACAACGAGTCCTCTTT 3′;

(SEQ ID NO:19)

5′ CGGTGCACAGGCTAGCTACAACGACAGCTTGCG 3′;

(SEQ ID NO:20)

5′ TCCGGAACAGGCTAGCTACAACGAAATGGCCAC 3′;

(SEQ ID NO:21)

5′ TCGTCTGTAGGCTAGCTACAACGACTGGCAGGT 3′;

(SEQ ID NO:22)

5′ ATCCGGTGAGGCTAGCTACAACGAGATCGTCTG 3′;

(SEQ ID NO:23)

5′ GCACAGCAAGGCTAOCTACAACGAGCGTCGAGG 3′;

(SEQ ID NO:24)

5′ GGGAAGGCAGGCTAGCTACAACGAAGCAATGCG 3′;

(SEQ ID NO:25)

5′ GCTTGGGGAGGCTAGCTACAACGAAGAAGCTGA 3′;

(SEQ ID NO:26)

5′ GTAAAGGGAGGCTAGCTACAACGAAGGGCTGGG 3′;

(SEQ ID NO:27)

5′ GAAACACCAGGCTAGCTACAACGAGGTGGGAAA 3′;

(SEQ ID NO:28)

5′ GGGGCAGGAGGCTAGCTACAACGATTGGGGAGG 3′;

(SEQ ID NO:29)

5′ CAGAGCTGAGGCTAGCTACAACGAACCATGGCT 3′;

(SEQ ID NO:30)

5′ GGACTGGGAGGCTAGCTACAACGAAGGGGCTGG 3′;

(SEQ ID NO:31)

5′ GGGCTAGCAGGCTAGCTACAACGATGGGACAGG 3′;

(SEQ ID NO:32)

5′ GGCCTCTGAGGCTAGCTACAACGAAGCGTTCCT 3′;

(SEQ ID NO:33)

5′ TCTTCATCAGGCTAGCTACAACGACAAACTGCA 3′;

(SEQ ID NO:34)

5′ AGTTGTCGAGGCTAGCTACAACGAGGATGCCAG 3′;

(SEQ ID NO:35)

5′ GGGGGGCCAGGCTAGCTACAACGAAGGTATGCC 3′;

(SEQ ID NO:36)

5′ CCATCAGCAGGCTAGCTACAACGAGGGCTCAGT 3′;
and

(SEQ ID NO:37)

5′ AGAAGTCCAGGCTAGCTACAACGAGTCCGCAAT 3′.

In a particularly preferred embodiment, the DNAzyme has the sequence 5′ GAGGGGGAAGGCTAGCTACAACGAAGTTCGTCC 3′ (SEQ ID NO: 4).

In applying DNAzyme-based treatments, it is preferable that the DNAzymes be as stable as possible against degradation in the intra-cellular milieu. One means of accomplishing this is by incorporating a 3′-3′ inversion at one or more termini of the DNAzyme. More specifically, a 3′-3′ inversion (also referred to herein simply as an “inversion”) means the covalent phosphate bonding between the 3′ carbons of the terminal nucleotide and its adjacent nucleotide. This type of bonding is opposed to the normal phosphate bonding between the 3′ and 5′ carbons of adjacent nucleotides, hence the term “inversion”. Accordingly, in a preferred embodiment, the 3′-end nucleotide residue is inverted in the binding domain contiguous with the 3′ end of the catalytic domain. In addition to inversions, the instant DNAzymes may contain modified nucleotides or nucleotide linkages. Modified nucleotides include, for example, N3′-P5′ phosphoramidate linkages, 2′-O-methyl substitutions and peptide-nucleic acid linkages. These are well known in the art.

In a second aspect the present invention provides a pharmaceutical composition comprising a DNAzyme according to the first aspect and a pharmaceutically acceptable carrier.

In the context of the present invention, administering the pharmaceutical compositions of the second aspect can be effected or performed using any of the various methods and delivery systems known to those skilled in the art. The administering can be performed, for example, intravenously, orally, via implant, transmucosally, transdermally, topically, intramuscularly, intra-articularly, subcutaneously or extracorporeally. In addition, the instant pharmaceutical compositions ideally contain one or more routinely used pharmaceutically acceptable carriers. Such carriers are well known to those skilled in the art. The following delivery systems, which employ a number of routinely used carriers, are only representative of the many embodiments envisioned for administering the instant composition.

Transdermal delivery systems include patches, gels, tapes and creams, and can contain excipients such as solubilizers, permeation enhancers (e.g., fatty acids, fatty acid esters, fatty alcohols and amino acids), hydrophilic polymers (e.g., polycarbophil and polyvinylpyrolidone), and adhesives and tackifiers (e.g., polyisobutylenes, silicone-based adhesives, acrylates and polybutene).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty-acid esters and derivatives, and hydrophilic polymers such as hydroxypropylmethylcellulose and hyaluronic acid).

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

Topical delivery systems include, for example, gels and solutions, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In the preferred embodiment, the pharmaceutically acceptable carrier is a liposome or a biodegradable polymer. Examples of liposomes which can be used in this invention include the following: (1) CellFectin, 1:1.5 (M/M) liposome formulation of the cationic lipid N,NI,NII,NIII-tetramethyl-N,NI,NII,NIII-tetrapalmitylspermine and dioleoyl phosphatidyl-ethanolamine (DOPE)(GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-trimethyl-ammoniummethylsulfate) (Boehringer Manheim); and (4) Lipofectamine, 3:1 (M/M) liposome formulation of the polycationic lipid DOSPA and the neutral lipid DOPE (GIBCO BRL).

Delivery of the nucleic acid agents described may also be achieved via one or more of the following vehicles:

(a) liposomes and liposome-protein conjugates and mixtures;

(b) polymer formulations such as polyethylenimine (PEI);

(c) a viral-liposome complex, such as Sendai virus;

(d) a peptide-nucleic acid conjugate; or

(e) a cholesterol-nucleic acid conjugate (where cholesterol is preferably conjugated to the 5′ terminus of the oligonucleotide).

In order to treat arthritis, for example, the DNAzymes of the present invention are preferably administered by direct injection in to inflamed joints, either as naked DNA in solution or in liposome complexes. Asthma is preferably treated by administering DNAzyme of the present invention by aerosol. Inflammatory vascular and bowel diseases are preferably treated by intraluminal administration.

In a third aspect, the present invention provides a method of inhibiting NF-κB activity in a cell which method comprises introducing into the cell a DNAzyme of the first aspect.

In a fourth aspect, the present invention provides a method of inhibiting NF-κB activity in a subject which method comprises administering to the subject a pharmaceutical composition of the second aspect.

In a fifth aspect the present invention provides a method of treating an inflammatory disease in a subject which method comprises administering to the subject a therapeutically effective dose of a pharmaceutical composition of the second aspect.

In a preferred embodiment of the fifth aspect, the inflammatory disease is selected from the group consisting of inflammatory arthritis, asthma, inflammatory bowel disease, septic shock and vasculitis. Preferably, the inflammatory arthritis is selected from the group consisting of rheumatoid arthritis, osteoarthritis and seronegative arthritis.

In a sixth aspect the present invention provides a method of treating atherosclerosis in a subject which method comprises administering to the subject a therapeutically effective dose of a pharmaceutical composition of the second aspect.

In a seventh aspect the present invention provides a method of treating cancer or leukaemia in a subject which comprises administering to the subject a therapeutically effective dose of a pharmaceutical composition of the second aspect.

Determining therapeutically effective doses of the instant pharmaceutical composition can be done based on animal data using routine computational methods. In one embodiment, the effective dose contains between about 0.1 mg and about 1 g of the instant DNAzyme. In another embodiment, the effective dose contains between about 1 mg and about 100 mg of the instant DNAzyme. In a further embodiment, the effective dose contains between about 10 mg and about 50 mg of the instant DNAzyme. In yet a further embodiment, the effective dose contains about 25 mg of the instant DNAzyme. A single therapeutically effective dose can be administered over time as a plurality of lesser doses.

In one embodiment of the fourth to seventh aspects, the method is performed in vivo. In another embodiment, the method is performed ex vivo.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

This invention will be better understood by reference to the Experimental Details that follow, but those skilled in the art will readily appreciate that these are only illustrative of preferred aspects of the invention. Additionally, throughout this application, various publications are cited. The disclosure of these publications is hereby incorporated by reference into this application to describe more fully the state of the art to which this invention pertains.

### EXAMPLE 1

### Design of DNAzyme Constructs

Two DNA constructs, designated ND1 and ND2, were designed based on the 10-23 catalytic motif (Santoro and Joyce, 1997) flanked by two substrate-recognition domains of 9 deoxynucleotides each. An inverted thymidine was placed at the 3-prime terminal end of the oligodeoxynucleotides. This exposes an apparent 5-prime end in order to make the constructs resistant to 3-prime exonuclease activity.

Construct ND1 is designed to cleave RelA(p65) messenger RNA at the AUG translation start site, between A80 and U81. Construct ND2 is designed to cleave RelA(p65) messenger RNA at the next available AU or GU site in the 3′ direction, that is, cleavage between G91 and U92. Their respective controls, ND1c and ND2c, contain randomised hybridisation arms. The control oligonucleotides possess the consensus 10-23 catalytic motif except for the alteration of a single base at the 5′ end of the catalytic motif. In ND1c there is an A to C change, which is not consistent with the general purpose catalytic motif. In ND2c there is an A to G change, which is consistent with catalytic activity (Santoro and Joyce, 1997).

The constructs are shown below, with hybridization arms underlined, inverted thymidines in parentheses (T) and the consensus 10-23 catalytic motif in bold.

| | | |
|---|---|---|
| ND1 | 5′ GTTCGTCC AGGCTAGCTACAACGA GGCCGGGGT (T) 3′ | (SEQ ID NO:3) |
| ND1c | 5′ GGTGACGC CGGCTAGCTACAACGA CTGCTGGTG (T) 3 | (SEQ ID NO:38) |

Re1A(p65) mRNA, target site for ND1, A80/A81

| | | | |
|---|---|---|---|
| 61 | 5′ CGCCCCCGGG ACCCCGGCCA UGGACGAACU GUUCCCCCUC AUCUUCCCGG-3′ | 110 | (SEQ ID NO:39) |
| ND2 | 5′ GAGGGGGA AGGCTAGCTACAACGA AGTTCGTCC (T) 3′ | | (SEQ ID NO:4) |
| ND2c | 5′ GTAGCATG GGGCTAGCTACAACGA TAGGGCAGC (T) 3′ | | (SEQ ID NO:40) |

Re1A(p65) mRNA, target site for ND2, G91/U92

| | | | |
|---|---|---|---|
| 61 | 5′ CGCCCCCGGG ACCCCGGCCA UGGACGAACU GUUCCCCCUC AUCUUCCCGG-3′ | 110 | (SEQ ID NO:39) |

### EXAMPLE 2

### In vitro Cleavage of a Synthetic RNA Target by DNAzymes ND1 and ND2

Oligonucleotides ND1, ND1c, ND2 and ND2c were incubated with RelA(p65) RNA (61-110) at 37° C. in 10 mM Mg²⁺ for the indicated times. The RNA was 32^{P} end-labelled prior to incubation with the DNAzymes. Cleavage to a single product of the expected molecular weights was observed for ND1 and ND2 (data not shown). There is no cleavage with the control oligonucleotides ND1c and ND2c. ND2 cleaves more efficiently than ND1.

### EXAMPLE 3

### Effects of DNAzymes ND2 on NF-κB and AP-1 Dependent Luciferase Reporter Gene in the Presence of a Liposome, CellFectin (Life Technologies)

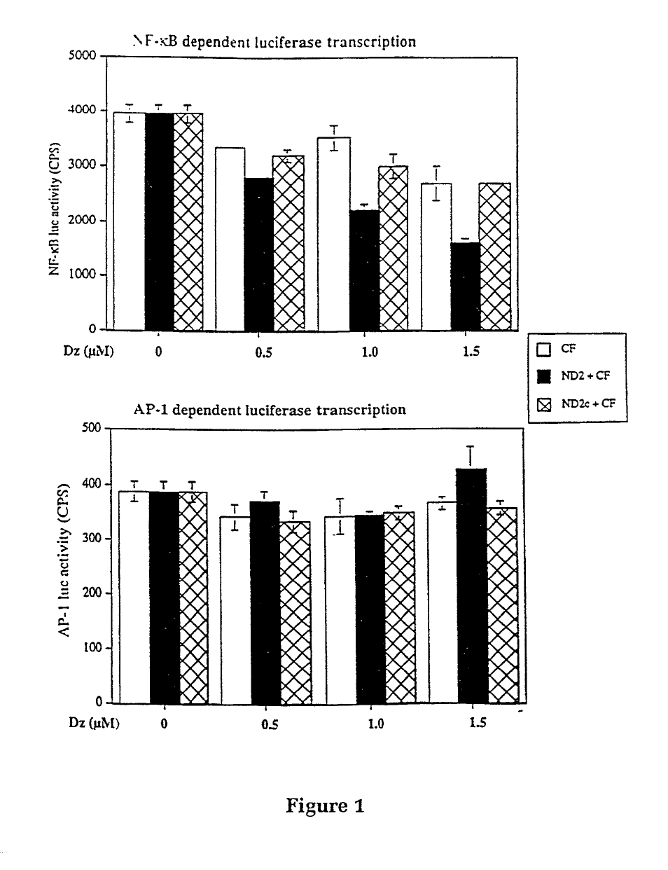

HeLa cells were stably transfected with plasmids containing a luciferase gene (Promega) transcribed from artificial promoters dependent on six NF-κB binding sites and three AP-1 sites. DNAzymes (Dz) were complexed with CellFectin at a ratio of DNAzyme 1 μM per CellFectin 2.5 ug/ml. After administration of Dz to HeLa cells, luciferase was induced with interleukin-1β 10 ng/ml. FIG. 1 shows that ND2 causes a concentration dependent inhibition of NF-κB dependent gene expression. Inhibition by ND2 is significantly greater than with the controls ND2c and vehicle alone, at all concentrations. Most importantly, there is no inhibition of AP-1 dependent gene expression by either ND2 or ND2c, indicating specificity of ND2 for the transcription factor NF-κB when compared to another inducible transcription factor.

### EXAMPLE 4

### Effect of DNAzyme (Dz) ND2 Compared to its Control ND2c on NF-κB Dependent Transcription

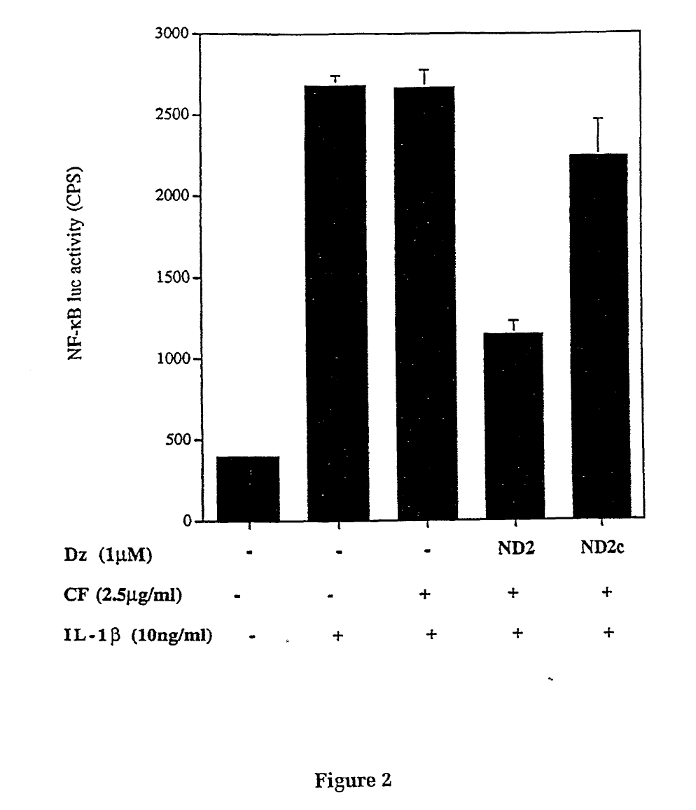

HeLa cells stably transfected with the NF-κB dependent luciferase reporter gene were treated with ND2/CellFectin, ND2c/CellFectin and CellFectin alone, and induced with interleukin-1β (IL-1β, 10 ng/ml). The presence of ND2 is necessary for specific inhibition of NF-κB gene expression. In multiple experiments there was approximately 40%-60% inhibition of inducible gene expression (FIG. 2).

### EXAMPLE 5

### Effect on DNAzymes (Dz) on NF-κB DNA Binding in HeLa Cells in the Presence of CellFectin

DNAzymes ND1 and ND2, and the control oligonucleotides ND1c and ND2c, were complexed with the liposome reagent CellFectin and used to treat HeLa cells. NF-κB DNA binding was induced with interleukin-1β (IL-1β, 10 ng/ml), nuclear extracts prepared from the cells and the extracts analysed by electrophoretic mobility shift analysis (EMSA) using NF-κB and AP-1 as the probes (data not shown). The indicated bands (p50/p65, p50/p50 and AP-1) have been characterised by supershift with antibodies and competition with unlabelled specific probes (not shown). The lower band in the NF-κB EMSA is non-specific. The only significant effects in these EMSAs are the induction of the p50/p65 NF-κB DNA binding by IL-1β and its inhibition back towards the uninduced state with ND2.

### EXAMPLE 6

### Selection of Additional Human RelA Cleaving DNAzymes

Previous results have shown that for any given sequence, usually only 10-20% of DNAzymes targeting purine-uracil (RU) sites are active against the full length substrate. The reasons for this are not well understood, however, it is thought that differences in DNAzyme-substrate hybridisation thermodynamics and RNA substrate folding (secondary structure) produce dramatic variations in the efficiency of DNAzyme catalysis. While nearest neighbour analysis of heteroduplex can be predictive of DNAzyme binding domain hybridisation thermodynamics, it is almost impossible to predict the activity of individual DNAzymes against long folded RNA substrates. The most reliable way to determine the RNA cleavage activity of different DNAzyme sites along the target RNA (such as RelA) is to test them all empirically. This very difficult, laborious and time consuming task often restricts the scope of this type of analysis. Accordingly a multiplex cleavage assay has been developed which allows high throughput cleavage analysis of all candidate DNAzymes across a range of concentrations in a single experiment (Cairns et al., 1999).

The human RelA mRNA sequence contains 126 RU dinucleotide sites which are potentially cleavable by the 10-23 DNAzyme. As only a portion of these sites were likely to be cleaved efficiently by DNAzymes under native conditions, a multiplex cleavage assay was emplyed to identify efficient cleavage sites. From the 126 possible sites about 30 were excluded from the cleavage assay as their sequences failed through computational analysis to reach minimum thermodynamic standards. These exclusions were made on the basis of two types of analysis; (1) nearest neighbour prediction of hybridisation free energy (Sugimoto et al.,1995), such that all binding domain-substrate heteroduplex had a predicted value of ΔGo<-10 kcal.mol-1, (2) DNAzyme oligonucleotide secondary structure (caused by internal or self complementarity) such that no oligonucleotides were used if they produced stable stem-loops or “hairpin” folds at a predicted melting temperature (Tm)=70° C. Another 8 DNAzyme sites were excluded as they were not contained within the transcript used in the assay.

The remaining 88 DNAzymes were synthesised and divided into six groups arranged according location on the RelA transcript. These were then incubated with radiolabelled transcript at three different concentrations. The products of this multiplex cleavage reaction were then analysed by primer extension reactions specific for each segment to reveal the active DNAzyme molecules. A phosphorimager was then used to determine the identity and intensity of respective DNAzyme cleavage bands. From these analyses the most active DNAzymes were chosen (Table 1).

TABLE 1
RelA target site selection using an in vitro multiplex
cleavage assay

| | | | | |
|---|---|---|---|---|
| Name | Sequence | SEQ ID NO. | Activity | Position |

| | | | | |
|---|---|---|---|---|
| DT923 | TGATCTCCAGGCTAGCTACAACGAATAGGGGcc | 5 | *** | G140 |
| DT925 | GCTGCTCAAGGCTAGCTACAACGAGATCTCCac | 6 | *** | A149 |
| DT927 | CGCCTGGGAGGCTAGCTACAACGAGCTGCCCgc | 7 | *** | A215 |
| DT928 | TTGGTGGTAGGCTAGCTACAACGACTGTGCTcc | 8 | *** | A237 |
| DT929 | TGATCTTGAGGCTAGCTACAACGAGGTGGGGtg | 9 | **** | A260 |
| DT933 | CCTTTCCTAGGCTAGCTACAACGAAAGCTCGtg | 10 | *** | G350 |
| DT939 | TTCTTCACAGGCTAGCTACAACGAACTGGATtc | 11 | *** | G438 |
| DT941 | TGGTCTGGAGGCTAGCTACAACGAGCGCTGAct | 12 | **** | A479 |
| DT942 | TAGTCCCCAGGCTAGCTACAACGAGCTGCTCtt | 13 | **. | G525 |
| DT946 | GGTCCCGCAGGCTAGCTACAACGATGTCACCtg | 14 | *** | G572 |
| DT947 | CCTGCCTGAGGCTAGCTACAACGAGGGTCCCgc | 15 | *** | A583 |
| DT955 | ACCTTGTCAGGCTAGCTACAACGAACAGTAGga | 16 | *** | G726 |
| DT956 | CTTTCTGCAGGCTAGCTACAACGACTTGTCAca | 17 | *** | G734 |
| DT957 | ACACCTCAAGGCTAGCTACAACGAGTCCTCTtt | 18 | **** | A749 |
| DT959 | CGGTGCACAGGCTAGCTACAACGACAGCTTGcg | 19 | **** | A807 |
| DT962 | TCCGGAACAGGCTAGCTACAACGAAATGGCCac | 20 | *** | G830 |
| DT971 | TCGTCTGTAGGCTAGCTACAACGACTGGCAGgt | 21 | *** | A951 |
| DT973 | ATCCGGTGAGGCTAGCTACAACGAGATCGTCtg | 22 | *** | G963 |
| DT981 | GCACAGCAAGGCTAGCTACAACGAGCGTCGAgg | 23 | **** | A1070 |
| DT982 | GGGAAGGCAGGCTAGCTACAACGAAGCAATGcg | 24 | **. | G1076 |
| DT983 | GCTTGGGGAGGCTAGCTACAACGAAGAAGCTga | 25 | *** | G1100 |
| DT984 | GTAAAGGGAGGCTAGCTACAACGAAGGGCTGgg | 26 | *** | A1125 |
| DT986 | GAAACACCAGGCTAGCTACAACGAGGTGCGAaa | 27 | *** | A1175 |
| DT988 | GGGGCAGGAGGCTAGCTACAACGATTGGGGAgg | 28 | **** | G1235 |
| DT991 | CAGAGCTGAGGCTAGCTACAACGAACCATGGct | 29 | *** | A1279 |
| DT992 | GGACTGGGAGGCTAGCTACAACGAAGOGGCTgg | 30 | **** | G1307 |
| DT993 | GGGCTAGGAGGCTAGCTACAACGATGGGACAgg | 31 | *** | G1313 |
| DT994 | GGCCTCTGAGGCTAGCTACAACGAAGCGTTCct | 32 | *** | G1387 |
| DT995 | TCTTCATCAGGCTAGCTACAACGACAAACTGca | 33 | **. | A1416 |
| DT998 | AGTTGTCGAGGCTAGCTACAACGAGGATGCCag | 34 | *** | G1484 |
| DT1001 | GGGGGGCCAGGCTAGCTACAACGAAGGTATGcc | 35 | *** | G1529 |
| DT1002 | CCATCAGCAGGCTAGCTACAACGAGGGCTCAgt | 36 | **. | A1553 |
| DT1008 | AGAAGTCCAGGCTAGCTACAACGAGTCCGCAat | 37 | *** | A1697 |

The *, **, *** rating was used to designate DNAzyme activity down to 500 nM, 50 nM and 5 nM concentration respectively. The **** rating was used to indicate very strong activity throughout the tested concentration ranges. The **. rating was used to indicate DNAzymes which displayed only very week activity at the 5 nM range relative to the higher concentrations tested. DNAzymes with ** rating and below were excluded from this list.

### CONCLUSIONS

The results presented in Examples 1 to 5 above demonstrate the following:

(1) DNAzymes ND1 and ND2 specifically cleave an RNA target at the expected sites.

(2) ND2 is more potent than ND1 and is therefore a preferred candidate as a therapeutic substance.

(3) ND2 specifically inhibits NF-κB dependent transcription in a concentration dependent manner. ND2 does not inhibit an unrelated inducible transcription factor, namely AP-1 and the control oligonucleotide ND2c does not inhibit NF-κB dependent transcription.

(4) ND2 specifically inhibits inducible binding of the NF-κB protein dimer p50/p65 in its DNA response element.

(5) A liposome reagent enhances inhibition of NF-κB dependent transcription in cell culture. It is recognised that the type of liposome will vary with cell type. It will be appreciated, however, that treatment of animal or human arthritic joints with DNAzymes in the absence of a liposomal reagent is possible.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES:

Auphan, N., DiDonato, J. A., Rosette, C., Helmberg, A., Karin, M. (1995) Science; 270:286-290.

Breaker, R. R. and Joyce, G. (1994) Chemistry and Biology 1:223-229.

Breaker, R. R. and Joyce, G. F. (1995) Chem. Biol. 2, 655-660.

Brinckerhoff, C. E. (1991) Arthritis Rheum; 34:1073-5.

Brostjan, C., Anrather, J. Csizmadia, V., Stroka, D., Soares, M., Bach, F. H., et al. (1996) J. Biol. Chem.; 271:19612-19616.

Cai Z, Korner M, Tarantino N, Chouaib S. (1997) IkBa overexpression in human breast carcinoma MCF7 cells inhibits nuclear factor-kB activation but not tumour necrosis factor-a-induced apoptosis. J Biol Chem; 272:96-101.

Caldenhoven, E., Liden, J., Wissink, S., Van de Stolpe, A., Raijmakes, J., Koenderman, L., et al. (1995) Mol. Endo.; 9:401-412.

Cairns, M. J., Hopkins, T. M., Witherington, C., Wang, L. and Sun, L. Q. (1999) Target site selection for an RNA-cleaving catalytic DNA. Nature Biotechnol. 17, 480-486.

Carmi, N., et al. (1996) Chemistry and Biology 3:1039-1046.

Chu, C. Q., Field, M., Feldmann, M., Maini, R. N. (1991) Arthritis Rheum; 34:1125-1132.

Frantz, B., Nordby, E. C., Bren, G., Steffan, N., Paya, C. V., Kincaid, R. L., et al. (1994) EMBO; 861-870.

Frantz, B., O'Neill, E. A. (1995). Science; 270:2017-2018.

Handel, M. L., Lehmann, T. P. (1998) Rheumatoid arthritis: Implications for future therapy Keystone Symposium; 405.

Handel, M. L., McMorrow, L. B., Gravallese, E. M. (1995a) Arthritis Rheum; 38:1762-70.

Handel, M. L., Watts, C. K. W., de Fazio, A., Day, R. O., Sutherland, R. L. (1995b) Proc. Natl. Acad. Sci. USA; 92:4497-4501.

Handel, M. L., Watts, C. K. W., Sivertsen, S., Day, R. O., Sutherland, R. L. (1996) Mol. Pharmacol.; 50:501-505.

Haseloff, J., Gerlach, W. L. (1988) Nature (334):585-591.

Higgins K A, Perez J R, Coleman T A, Dorshkind K, McComas W A, Sarmiento U M, et al. (1993) Antisense inhibition of the p65 subunit of NF-kB blocks tumorigenicity and causes tumor regression. Proc Natl Acad Sci USA; 90:9901-5.

Kamei, Y., Xu, L., Heinzel, T., Torchia, J., Kurokawa, R., Gloss, B., et al (1996) Cell; 85:403-414.

Kashani-Sabet, M., et al. (1992) Antisense Research and Development 2:3-15.

Kinne, R. W., Boehm, S., Iftner, T., Aigner, T., Vornehm, G., Weseloh, G. et al. (1994) Scand J Rheumatol; 23 (supplement 101):111-5.

Kitajima I, Shinohara T, Bilakovics J, Brown D A, Xu X, Nerenbergt M. (1992) Ablation of Transplanted HTLV-I Tax-Transformed Tumors in Mice by Antisense Inhibition of NF-κB. Science; 258:1792-5.

Koizumi, M., et al. (1989) Nucleic Acids Research 17:7059-7069.

Kopp, E., Ghosh, S. (1994) Science; 265:956-959.

Miagkov, A. V., Kovalenko, D. V., Brown, C. E., Didsbury, J. R., Cogswell, J. P., Stimpson, S. A. et al. (1998) Proc. Natl. Acad. Sci. USA; 95:13859-64.

Otsuka, E. and Koizumi, M., Japanese Patent No. 4,235,919.

Pan, T. and Uhlenbeck, O.C. (1996) Biochemistry 31:3887-3895.

Poole, B., Ohkuma, S. (1981) J. Biol. Chem.; 90:665-669.

Raillard, S. A. and Joyce, G. F. (1996) Biochemistry 35:11693-11701.

Ray, A., Prefontaine, K. E. (1994) Proc. Natl. Acad. Sci. USA; 91:752-756.

Santoro, S. W., Joyce, G. F. (1997) Proc. Natl. Acad. Sci. USA 1997; 94:4262-4266.

Scheinman, R. I., Cogswell, P. C., Lofquist, A. K., Baldwin Jr A.S. (1995) Science; 270:283-286.

Schutze, S., Weigmann, K., Machleidt, T., Krone, M. (1995) Immunobiol.; 193:193-203.

Shaw III C. F. (1979) Inorg. Perspect. Biol. Med.; 2:287-355.

Stuhlmeier, K. M., Kao, J. J., Bach, F. H. (1997) J. Clin. Invest.; 100:972-985.

Sugimoto, N., Nakano, S., Katoh, M., Matsumura, A., Nakamuta, H., Ohmichi, T., Yoneyama, M., and Sasaki, M. (1995) Thermodynamic parameters to predict stability of RNA/DNA hybrid duplexes. Biochemistry 34, 11211-11216.

Sun, L. Q., et al. (1997) Mol. Biotechnology 7:241-251.

Symons, R. H. (1992) Annu. Rev. Biochem. 61, 641-671.

Tsang, J. and Joyce, G.F. (1994) Biochemistry 33:5966-5973.

Wang C Y, Cusack J C, Liu R, Baldwin A S. (1999) Control of inducible chemoresistance: Enhanced anti-tumor therapy through increased apoptosis by inhibition of NF-κB. Nature Medicine; 5:412-7.

Weigmann, K., Schutze, S.,. Machleidt, T., Witte, D., Kronke, M. (1994) Cell; 78:1005-1015.

Yang, J. P., Merin, J. P., Nakano, T., Kato, T., Kitade, Y., Okamato, T. (1995) FEBS Letters; 361:89096.

1. A DNAzyme which specifically cleaves RelA(p65) mRNA, the DNAzyme comprising
(i) a catalytic domain which cleaves mRNA at a purine:pyrimidine cleavage site;
(ii) a first binding domain contiguous with the 5′ end of the catalytic domain; and
(iii)a second binding domain contiguous with the 3′ end of the catalytic domain,
wherein the binding domains are sufficiently complementary to the two regions immediately flanking a purine:pyrimidine cleavage site within the region of RelA(p65) mRNA corresponding to nucleotides 1 to 1767 as shown in SEQ ID NO: 1, such that the DNAzyme cleaves the RelA(p65) mRNA.

2. A DNAzyme as claimed in claim 1 wherein each binding domain is nine or more nucleotides in length.

3. A DNAzyme as claimed in claim 1 or claim 2 in which the catalytic domain has the nucleotide sequence GGCTAGCTACAACGA (SEQ ID NO: 2).

4. A DNAzyme as claimed in any one of claims 1 to 3 in which the cleavage site corresponds to a site selected from the group consisting of:
(i) the AT site at nucleotides 80-81;
(ii) the GT site at nucleotides 91-92;
(iii)the GT site at nucleotides 140-141;
(iv) the AT site at nucleotides 149-150;
(v) the AT site at nucleotides 215-216;
(vi) the AT site at nucleotides 237-238;
(vii) the AT site at nucleotides 260-261;
(viii)the GT site at nucleotides 350-351;
(ix) the GT site at nucleotides 438-439;
(x) the AT site at nucleotides 479-480;
(xi) the GT site at nucleotides 525-526;
(xii) the GT site at nucleotides 572-572;
(xiii)the AT site at nucleotides 583-584;
(xiv) the GT site at nucleotides 726-727;
(xv) the GT site at nucleotides 734-735;
(xvi) the AT site at nucleotides 749-750;
(xvii)the AT site at nucleotides 807-808;
(xviii) the CT site at nucleotides 830-831;
(xix) the AT site at nucleotides 951-952;
(xx) the AT site at nucleotides 963-964;
(xxi) the AT site at nucleotides 1070-1071;
(xxii) the GT site at nucleotides 1076-1077;
(xxiii) the GT site at nucleotides 1100-1101;
(xxiv) the AT site at nucleotides 1125-1126;
(xxv) the AT site at nucleotides 1175-1176;
(xxvi) the GT site at nucleotides 1235-1236;
(xxvii) the AT site at nucleotides 1279-1280;
(xxviii)the GT site at nucleotides 1307-1308;
(xxix) the AT site at nucleotides 1313-1314;
(xxx) the GT site at nucleotides 1387-1388;
(xxxi) the AT site at nucleotides 1416-1417;
(xxxii) the GT site at nucleotides 1484-1485;
(xxxiii)the AT site at nucleotides 1529-1530;
(xxxiv) the AT site at nucleotides 1553-1554; and
(xxxv) the AT site at nucleotides 1697-1698.

5. A DNAzyme as claimed in claim 4 in which the cleavage site corresponds to the GT site at nucleotides 91-92.

6. A DNAzyme as claimed in claim 1 which has a sequence selected from the group consisting of:
(SEQ ID NO:3);
5′ GTTCGTCCAGGCTAGCTACAACGAGGCCGGGGT 3′
(SEQ ID NO:4);
5′ GAGGGGGAAGGCTAGCTACATACAAGTTCGTCC 3′
(SEQ ID NO:5);
5′ TGATCTCCAGGCTAGCTACAACGAATAGGGGCC 3′
(SEQ ID NO:6);
5′ GCTGCTCAAGGCTAGCTACAACGAGATCTCCAC 3′
(SEQ ID NO:7);
5′ CGCCTGOGAGGCTAGCTACAACGAGCTGCCCGC 3′
(SEQ ID NO:8);
5′ TTGGTGGTAGGCTAGCTACAACGACTGTGCTCC 3′
(SEQ ID NO:9);
5′ TGATCTTGAGGCTAGCTACAACGAGGTGGGGTG 3′
(SEQ ID NO:10);
5′ CCTTTCCTAGGCTAGCTACAACGAAAGCTCGTG 3′
(SEQ ID NO:11);
5′ TTCTTCACAGGCTAGCTACAACGAACTGGATTC 3′
(SEQ ID NO:12);
5′ TGGTCTGGAGGCTAGCTACAACGAGCGCTGACT 3′
(SEQ ID NO:13);
5′ TAGTCCCCAGGCTAGCTACAACGAGCTGCTCTT 3′
(SEQ ID NO:14);
5′ GGTCCCGCAOGCTAGCTACAACGATGTCACCTG 3′
(SEQ ID NO:15);
5′ CCTGCCTGAGGCTAGCTACAACGAGGGTCCCGC 3′
(SEQ ID NO:16);
5′ ACCTTGTCAGGCTAGCTACAACGAACAGTAGGA 3′
(SEQ ID NO:17);
5′ CTTTCTGCACGCTAGCTACAACGACTTGTCACA 3′
(SEQ ID NO:18);
5′ ACACCTCAAGGCTAGCTACAACGAGTCCTCTTT 3′
(SEQ ID NO:19);
5′ CGGTGCACAGGCTAGCTACAACGACAGCTTGCG 3′
(SEQ ID NO:20);
5′ TCCGGAACAGGCTAGCTACAACGAAATCGCCAC 3′
(SEQ ID NO:21);
5′ TCGTCTGTAGGCTAGCTACAACGACTGGCAGGT 3′
(SEQ ID NO:22);
5′ ATCCGGTGAGGCTAGCTACAACGAGATCGTCTG 3′
(SEQ ID NO:23);
5′ GCACAGCAAGGCTAGCTACAACGAGCGTCGAGG 3′
(SEQ ID NO:24);
5′ GGGAAGGCAGGCTAGCTACAACGAAGCAATGCG 3′
(SEQ ID NO:25);
5′ GCTTGGGGAGGCTAGCTACAACGAAGAAGCTGA 3′
(SEQ ID NO:26);
5′ GTAAAGGGAGGCTAGCTACAACGAAGGGCTGGG 3′
(SEQ ID NO:27);
5′ GAAACACCAGGCTAGCTACAACGAGGTGGGAAA 3′
(SEQ ID NO:28);
5′ GGGGCAGGAGGCTAGCTACAACGATTGGGGAGG 3′
(SEQ ID NO:29);
5′ CAGAGCTGAGGCTAGCTACAACGAACCATGGCT 3′
(SEQ ID NO:30);
5′ GGACTGGGAGGCTAGCTACAACGAAGGGGCTGG 3′
(SEQ ID NO:31);
5′ GGGCTAGGAGGCTAGCTACAACGATGGGACAGG 3′
(SEQ ID NO:32);
5′ GGCCTCTGAGGCTAGCTACAACGAAGCGTTCCT 3′
(SEQ ID NO 33);
5′ TCTTCATCAGGCTAGCTACAACGACAAACTCCA 3′
(SEQ ID NO:34);
5′ AGTTGTCGAGGCTAGCTACAACCAGGATGCCAG 3′
(SEQ ID NO:35);
5′ GGGGGCCCAGCCTAGCTACAACGAAGGTATCCC 3′
(SEQ ID NO:36);
5′ CCATCAGCAGGCTAGCTACAACGAGGGCTCAGT 3′
and
(SEQ ID NO:37).
5′ AGAAGTCCAGGCTAGCTACAACGAGTCCGCAAT 3′

7. A DNAzyme as claimed in claim 6 which has the sequence 5′ GAGGGGGAAGGCTAGCTACAACGAAGTTCGTCC 3′.

8. A DNAzyme as claimed in any one of claims 1 to 7, wherein the 3′-end nucleotide residue is inverted in the binding domain contiguous with the 3′ end of the catalytic domain.

9. A pharmaceutical composition comprising a DNAzyme according to any one of claims 1 to 8 and a pharmaceutically acceptable carrier.

10. A method of inhibiting NF-κB activity in a cell which method comprises introducing into the cell a DNAzyme of any one of claims 1 to 8.

11. A method of inhibiting NF-κB activity in a subject which method comprises administering to the subject a pharmaceutical composition of claim 9.

12. A method of treating an inflammatory disease in a subject which method comprises administering to the subject a therapeutically effective dose of a pharmaceutical composition of claim 9.

13. A method as claimed in claim 12, wherein the inflammatory disease is selected from the group consisting of inflammatory arthritis, asthma, inflammatory bowel disease, septic shock and vasculitis.

14. A method as claimed in claim 13, wherein the inflammatory arthritis is selected from the group consisting of rheumatoid arthritis, osteoarthritis and seronegative arthritis.

15. A method of treating atherosclerosis in a subject which method comprises administering to the subject a therapeutically effective dose of a pharmaceutical composition of claim 9.

16. A method of treating cancer or leukaemia in a subject which comprises administering to the subject a therapeutically effective dose of a pharmaceutical composition of claim 9.

17. A method as claimed in any one of claims 10 to 15, wherein the method is performed in vivo.

18. A method as claimed in any one of claims 10 to 15, wherein the method is performed ex vivo.
